SEQUENCE LISTING (2 sequences)

SEQ ID NO: 1 - DNA, 6877 bp, Solanium lycopersicum
  misc_feature (4386)..(4386): n is a, c, g, or t
aataattagtagattccaaagatataataatatggagaaattcaatgaagaagaagatcaagctaaggttacaacaattaatgtggatagctttggagctaaaggtgatggaagtatagatgatacaaatgtaagtattagttttcttttaaaaaaaaattctctttcgaactattattaaaaacacgtgaatttcttatgaaaatttctcagacaaatcatcgtatattgacttgttaaaagcttttcagacagacacgcttccattcaaaaaaaataattaaaattcacatgtttttaatagtgaaacaatatatgtatactaccttgatttgatttaatttatttggcgatatttgatttaagttaaaattaaaaataaatgaagattttaaaacttatgatgttaattagatgtcctataatttttcaatggttgtaaaggcttaaatgtatatataaaaataggatgtatataaagttacattattttaaaatataaataaaatatttttattagtcatacatacaaaatgaaataaaagaagtatataaagttacattattttaaaatataaataaaatatttttattagtcatacatataaaatgaaataaaagaagtatattattagatataaaagtttgtgaagaaatgcttttttttttaaaaagaatttaatttgtatgcaacgataagttgataactgataacatatgtcacatctttgcgggttgatttgcattagttattggtgttaaaatcaaataaatcaatttatccggtcttataattattaaattaaagtaaatctaaaataatatatatttatcgatttgattgttattggtttcagtttgattagattatttgattactaatcatatataaaattaaaagaaataatttattaaaaacgtgaaaagagcggtaacaatttattcaaaatgaaaaatagttagaatgactaaaattaaataattttcaattacagaatttactatgaataaagcttcaaaatttactattttgacgtagaagaaagagacaataacattttttgtcttaaaaaaatgatcataacggttatatacatgaaaccaatataagtcaaatgttctcaccttgaggattattgtttttgcgtaagtacaaatcataaattttaatgaaaacaaatataatatctttaaaaatcgtctacaaaaataatatattatatatgtatgataaaatataatatatgtatatgatttatcgatttgattcagttatttctttgatatttttctagcaaaatcacaatcaaactaaatactattgatttttaaaaatccaaaatcaaatcaaaccataatccaaattcaatttgtatcaattttatccaaacgtgaacactcctagtcacataacttaaaaattaattataagtaaccatcaataagaaatgaccgaactagttaattaatataagtatacacacacattaattaattaaacatcttttttcattcgatattgatattgaagctcaattgtttcgactttgtcctgcgtaaggcctcatttggcaaaaaaatactctttagcaagaattttttttatatgcaaaactcaaatctgagactccgaattaagataggagaattaaacatctcatttcttggtggaaaataaagattatgtcatacttagaagccaattattatcatggtgaatatatgtagcaggttgggattacttctacgacagaattttatcagcatcataagcaatttaattagttttttttaataaattaattaatatattaaaataaatttatcttaatttcaggcatttcaaaaagcatggaaagaagcttgttcatcttcacatgttgtgaattttgtggtgtcccagaacaagaaatatcttctcaaaccaatcaaattttatgggccatgcaaatcttccattacaatgcaggttaggttgacatatcattatgtaaaaaaaaaactcgaagtaacgataaaattattctcgtcctaatcactataaagtatggatgttctgagtcatggtcaagtaattagatcttgaggcttgcatcagggtaggctgtttgtgtcatggtgtggctctttccaaaacttatatgaaagtgagatgctttgtgcatccgattatcgattcaaactaacagaataaaagttttttttatacgattaggttattttaaaatatgataaataatcttattcgacgagttgaattgcatcgtgtatgtggatctatagtgtaatctaaacatttacgcgactttattgaatagtatccaaaaaatattaaatcagaaattgatattgttatataaacggataagtttcaaatcttgaatacacctttgataatatgaaaaacttattacattgtctgcgcacggatataagttaattaaactctgctattattatatatatgtagagaaataagctctatgtatagtgacaaagctatatccgttgaacgatgattagttgaacacttaggttaaaatataaccttaattttatatattgcgacacctttcgtgaaatttttaaatctcgtcaccacttttagttacatttggaaataaataataggttggagtgtagaatattattttgacacttttgttcaatttttttttattgtagatttatggaaccctattagcatctgatgatacttcagattacaagaaggatagtaggcactggcttatttttgatagtgttcaaaaattggttgttggaggagctggagttatcaatggcaatggcaaaatttggtggcaacattcttgcaaaattaataaaaaattggtataaatacatacttaattttgaaagggtcgagaatcttgaaacaatcctaaagtttataggtcaccgtcagttcgaatcgtgtgcaaataattactattatttgcactaggatagactgtctacattatacgccaatcaagtgcaaccttcccaaaccttgcataagtgcatgatgttttatgagccggggccggaaagcccttttgtcctttacatacttaattttaaaaggagagtcttggagtaatgataaaattgtctatgtgcatgtgattataggttactgtccgttcaatccgtgtaggaacaattactaatacatgttttagaatagactatctatattacactccaatcaagtgcagttctttccgaaccttgcataaacacatgatgttttatgcaacggggcgtgggcaacccttttgccctttgcaaactttatttatttatttatatatatattcctatttgtttcgtttgtttttatgatatataactaattttgtctaatttttattcgtattgtctacttttttttttattttgcagccatgcaaggtagcacccacggtacgtttatttgatttgatttcttatttttcaaaagaacaaaacaattaattattacgtcatttttctttaatattttttttgttgatagagattttttattttattttattggtgacatttaaattttttcaggccctgacattttacaagtgtaacaacttgaaagtgaaggaccttaaaatagaaaatgcacaacaaatacatttgctaattgagaagtgtgttggtgttgaagttacaaaattggtagtgacttctccagaaaatagccctaatactgatggaatccatataactagcactcaaaatattcaaatttctgattccaccattgccacaggtatcgataaattaacgattttaatttttatatacttatagtaaaaaataattatattatcaagttgtatgtataaattaaaggtaaacatgcttttaataagtgaaacttatgataagttattagcctgaaaatatgaaagattaaaattttcgacaattgatcatcatcgtctcatcgaccacttggtctttgacgtagggcttttcctaattcccgaatgaacttgataagtcaactcaaagataaatatatgtttagatcatgagtaacgtgtgataccttaggtaaatttcacttcgatatacatataagaaatgttggtgtataggaaaacacaatttagacaatagtgtcaatgtggtttgatttaaaaggtgcgattggtataaaggaaaacatttttcagaatcaactcattttcctcaaaattaaggataatgaattcccttcaaaaattgaggaaaacatttttcaaaactctcatccaatttgaaattatatattttttaaagaaaacatcgatttcgaaaaatatattcaatttcaaaattttatatattcaccaccaccccaccccacccgatcatgccttcacccccccccccccccanaaaaaaaatagattttttttttaaaaaaatatttttaatttcaaaatttcatttctccatccctaccctttaccccccccccctaccaaccctctacccccctcccaaaaaagaaaattaagtttgtcttaaaaaatattttcgacttcaaatttttattttttcacccttacctcgacccccgccccccaacacccaccccaccccactccatctttaaagataaattttcattttgtttttttaaaaatatttttaacctcaaatttttattttttctacccctacgtccccctctccagtccccttaccatccctccagccccaccaaaaaaatatactttaatttttaaaaaatattttcaactttaaatttttattttttcactcctaacctcccgcacccacccacccccgccagccccccaccccccaaaaaaaattattttcaatttcaaaaattagtttgtactctagtaaaaataaaagatatttctcaaaaatatttttcattcataaattaaacactaaaaaatattttctactcatcaaccaaacatgaaaaaacaaatcataaatctacttattttctaaaaaaaaaacattttcctaattaccaaacacaccgaaaatcttgaaagtgtagttccaagcggataattaatgtcactatagtgagttgtactattatagattgttgaaataatatttacaaatattaaattatactaattgtacccaaaaaaaatgacattaaatttttttatttactaactatgtattttggaaataatttttgtggtaggtgatgattgcatctcaattgtggatggatctcagaaggtcttagccactggcattacttgtggaccaggtcatggaattaggtaaaaacaaaataatttcattatattagtattattatgctagtattatcttattttttattatattattagtgttatttctctaatttttattatctttactttcattacttttctttctttaatatctttatcattttatttttgtatatcttttgaactgttctgaaaaaattgtttttttatcgtttatatgaaacaatctcctaccccaccaaggtaagaaataagtttttgggtcaaacctcacttgcatgattattttctttatcatattcagattattttatcaaaatttataatttaattatgtaatttttttttattattattatttgtgcttagtattggaagtttgggaggtggaaattcagaagctcatgtgtctgatattcatgtaaatggagctaagctttatgaaactacaaatggacttaggattaagacttggccggtaaattaattaattaattatttttattaatttacaaatattctttataattaatatacatataattatattcttgaattattttgtatagggaggatttggaagtgcaagcaatattaagtatcaaaatgtggttatgaataatgtcaaaaatccaataattatagaccaaaattattgtgatcaagctgatggtccatgcaaagctgaggtaattgaaaatgaatttaacttatatatattaactatacgaaaaaataatctactttgttaaagcagatatcttatcgtattttttagataattaatccgttccattatgggttgttagttaagctatctttagatagccaaatatggtaaattttttaataatgtcgacatatgtatgttataaaaatatataacttaaactcgaacaaaaatgaattttatttatataacatacttaatgtatagaatatttttataccattagtgcaatttgctacatcatttggacaatatagtgttttaacttataaacaaaaataatttctccccttcaactttcttaaaattcaataaaatccccatacaaaatattgtataatcaaatttaccccactttgaaaatgtaacatataacgacatattgtacattataaagttgtgacggaatatctatgacgataatattaatgacagagttaatatggtcgattcataatatgattcaatcacaaacttgtgataatttcattgatctcttaatcacaaaaatatatttatacatttatagactgactcggcagttgaagtgaaaaatgtgatttatcaaaatatcaaaggcacaagtgcaacaaatgatgcaataagtatcaagtgcagcaaaaaaattccatgtgaaggaattttgatggagaatgtgaaattgttaggaggaaatggtgaaactccaaatggtatttggggaaatatcaataatcttacgtgcaaaaatgttttaccagaatgtcaaaaaaactcaaaaattgtataattaagtttgatagagttaattaatgtttaataggatattgtatagttatacatatgacaaattaagtgttagaatacataattagcaaatgatttgtgataaatgatattctttgtacacatgaaataa

SEQ ID NO: 2 - protein, 319 aa, Solanium lycopersicum
CSSSHVVNFVVSQNKKYLLKPIKFYGPCKSSITMQIYGTLLASDDTSDYKKDSRHWLIFDSVQKLVVGGAGVINGNGKIWWQHSCKINKKLPCKVAPTALTFYKCNNLKVKDLKIENAQQIHLLIEKCVGVEVTKLVVTSPENSPNTDGIHITSTQNIQISDSTIATGDDCISIVDGSQKVLATGITCGPGHGISIGSLGGGNSEAHVSDIHVNGAKLYETTNGLRIKTWPGGFGSASNIKYQNVVMNNVKNPIIIDQNYCDQADGPCKAETDSAVEVKNVIYQNIKGTSATNDAISIKCSKKIPCEGILMENVKLLGG

(19) United States
(12) Patent Application Publication
(10) Pub. No.: US 2010/0199374 A1
(43) Pub. Date: Aug. 5, 2010

(54) NOVEL POLYGALACTURONASES AND THEIR USES

(75) Inventors: Adriaan Willem Van Heusden, Wageningen (NL); Benoit Gorguet, Avignon (FR)

Correspondence Address: MORGAN LEWIS & BOCKIUS LLP, 1111 PENNSYLVANIA AVENUE NW, WASHINGTON, DC, 20004, US

(73) Assignee: WESTERN SEED INTERNATIONAL B.V., Naaldwijk (NL)

(21) Appl. No.: 12/670,825

(22) PCT Filed: Jun. 5, 2008
(86) PCT No.: PCT/NL08/50352

§ 371 Date: Jan. 26, 2010

Related U.S. Application Data
(60) Provisional application No. 60/952,046, filed on Jul. 26, 2007.

(30) Foreign Application Priority Data
Jul. 26, 2007 (EP) 07113263.3

(51) Int. Cl. A01H 1/00; C07H 21/04; C12Q 1/68; C12N 15/82

(52) U.S. Cl. 800/267; 536/23.2; 435/6; 435/468

(57) ABSTRACT

The present invention relates to nucleotide sequences of novel Solanum polygalacturonases. The nucleotide sequences may be used in marker assisted breeding, TILLING or in transgenic plants for the production of plants with a positional sterile phenotype due to non-dehiscent anthers.

### FIELD OF THE INVENTION

The present invention relates to the field of plant breeding, in particular the breeding of tomato plants. The invention extents to the fields of both classical and molecular plant genetics and relates to sequences of novel polygalacturonases and their use in marker assisted breeding or in transgenic plants, e.g. to produce plants with a positional sterile phenotype due to non-dehiscent anthers.

### BACKGROUND OF THE INVENTION

In higher plants mature pollen are released from the anther by dehiscence, which consists of a succession of cell destructions occurring successively in the tapetum, the septum and ultimately in the stomium. After degeneration of the tapetum, endothecium cells enlarge and lignified fibrous bands are deposited creating a thickening of the cell walls. Subsequently, dehydration and shrinkage of the endothecium and connective cells surrounding the locules create a breaking force in the stomium that eventually leads to pollen release (Keijzer 1987).

Recent progress was made in understanding the molecular control of anther dehiscence, which involves mainly the discovery of the implication of Jasmonic Acid (JA) and ethylene. Several mutants affecting diverse steps in the synthesis of JA in the anthers were identified in Arabidopsis. The observed phenotypes resulted in delay of anther dehiscence (reviewed by Scott et al, 2004). The role of ethylene signalling in this phenomenon was highlighted by Rieu et al. (2003) who observed a delay in the dehiscence of anthers of ethylene insensitive tobacco plants.

Polygalacturonases (PGs) belong to one of the largest hydrolase families (Torki et al, 2000; Markovic and Janecek, 2001). PGs activities have been shown to be associated with a wide range of plant developmental programs (reviewed by Hadfield and Bennett 1998), among them, anther dehiscence: Activity of PGs has been observed in the dehiscence zone of anthers of maize, tobacco, oilseed rape and Arabidopsis (Dubald et al 1993; Sander et al 2001). However their role in the dehiscence process has never been studied into details.

We have recently fine mapped the positional sterility-2 gene, conferring non-dehiscent anthers in tomato (Gorguet et al 2006). It is an object of the present invention to provide for the nucleotide and amino acid sequences of the ps-2 gene, as well as methods wherein these sequences are used in marker assisted breeding or in transgenic plants, e.g. to produce plants with a positional sterile phenotype due to non-dehiscent anthers and/or to alter fruit ripening.

### DESCRIPTION OF THE INVENTION

### Definitions

In this description, unless indicated otherwise, the terms and definitions used herein are those used in (Mendelian) genetics, for which reference is made to M. W. Strickberger, Genetics, second Edition (1976), in particular pages 113-122 and 164-177. As mentioned therein, “gene” generally means an inherited factor that determines a biological characteristic of an organism (i.c. a tomato plant), an “allele” is an individual gene in the gene pair present in the (diploid) tomato plant. In this context it is understood that the term a ps-2-gene or -allele as used herein refer to an allele of the a Dehiscence Polygalacturonase (DPG), that is capable of producing or contributing to the positional sterility phenotype of the invention. A ps-2-gene or -allele as used herein may thus refer to any loss of (DPG-)function allele that is capable of producing or contributing to the phenotypes of the invention of positional sterility and/or non-dehiscent anthers. A preferred example of a DPG gene is the Tomato Dehiscence Polygalacturonase (TDPG) gene described herein. A preferred example of a ps-2-gene or -allele is the particular tomato ps-2 allele described herein that has a C as last nucleotide of the 3′ end of the fifth exon of the TDPG gene.

A polygalacturonase (EC 3.2.1.15) is herein understood as an enzyme that katalyses the random hydrolysis of 1,4-alpha-D-galactosiduronic linkages in pectate and other galacturonans. Polygalacturonases are also referred to as pectin depolymerases or pectinases. Polygalacturonase activity is determined quantitatively by means of colorimetric detection of reducing sugar release as described previously (Parenicova et al., 1998, Eur. J. Biochem. 251:72-80), whereby 1 unit of polygalacturonase activity is defined as the amount of enzyme capable of releasing 1 μmole of reducing sugar ends per minute from polygalacturonic acid (Sigma) as the model substrate at 30° C. in 50 mM sodium acetate buffer, pH 4.2, with 0.25% (w/v) substrate concentration.

A plant is called “homozygous” for a gene when it contains the same alleles of said gene, and “heterozygous” for a gene when it contains two different alleles of said gene. The use of capital letters indicates a dominant (form of a) gene and the use of small letters denotes a recessive gene: “X,X” therefore denotes a homozygote dominant genotype for gene or property X; “X,x” and “x,X” denote heterozygote genotypes; and “x,x” denotes a homozygote recessive genotype. As commonly known, only the homozygote recessive genotype will generally provide the corresponding recessive phenotype (i.e. lead to a plant that shows the property or trait “x”) whereas the heterozygotic and homozygote dominant genotypes will generally provide the corresponding dominant phenotype (i.e. lead to a plant that shows the property or trait “X”), unless other genes and/or factors such as multiple alleles, suppressors, codominance etc. (also) play a role in determining the phenotype.

As a general rule, hybrid seed is obtained by crossing two different parent tomato plants, which most often belong to different lines. Using cultivation techniques and plant breeding techniques known per se, such hybrids can be provided with highly specific, desired properties, which makes it possible to “design” the hybrids, i.e. to confer to the hybrid plants predetermined inheritable characteristics. This is usually achieved by suitably choosing (the properties of) the two parent lines which are crossed to provide the hybrid seed. These are usually inbred lines, obtained by self-fertilization (self-pollination) over several generations, and such inbred lines will usually again have been specifically “designed” by the breeder so as to provide hybrid offspring with the desired properties, when crossed with another—usually predetermined—inbred parent line. As a rule, such parent lines will be genetically homozygote and identical (i.e. as a result of inbreeding) so that they can provide, in a stable and reliable manner, genetically uniform—albeit heterozygote—hybrid line combinations, which can combine the properties of the parent lines. In doing so, the aim is on the one hand to cross certain properties from the parent lines as purely as possible into the seed, while on the other hand use is made of the known effect of heterosis or inbred growth, which can provide improved properties regarding—inter alia—the growth of plants and fruits and thereby of the yield. This heterosis effect is obtained when/because the parent lines used are not related with respect to certain genetic properties (i.e. when the parent lines genetically “lie far apart”). For a further description of plant breeding techniques in general, and tomatoes in particular, using classical cultivation techniques, including the formation of hybrids, reference is made to the known handbooks, the contents of which are incorporated herein by reference.

As used herein, the term “plant” includes the whole plant or any parts or derivatives thereof, such as plant cells, plant protoplasts, plant cell tissue cultures from which tomato plants can be regenerated, plant calli, plant cell clumps, and plant cells that are intact in plants, or parts of plants, such as embryos, pollen, ovules, fruit (e.g. harvested tomatoes), flowers, leaves, seeds, roots, root tips and the like.

Botanical terminology: Linnaeus is considered the father of botanical classification. Although he first categorized the modern tomato as a Solanum, its scientific name for many years has been Lycopersicon esculentum. Similarly, the wild relatives of the modern tomato have been classified within the Lycopersicon genus, like L. pennellii, L. hirsutum, L. peruvianum, L. chilense, L. parviflorum, L. chmielewskii, L. cheesmanii, L. cerasiforme, and L. pimpinellifolium. Over the past few years, there has been debate among tomato researchers and botanists whether to reclassify the names of these species. The newly proposed scientific name for the modern tomato is Solanum lycopersicum. Similarly, the names of the wild species may be altered. L. pennellii may become Solanum pennellii, L. hirsutum may become S. habrochaites, L. peruvianum may be split into S. ‘N peruvianumr’ and S. ‘Callejon de Huayles’, S. peruvianum, and S. corneliomuelleri, L. parviflorum may become S. neorickii, L. chmeilewskii may become S. chmielewskii, L. chilense may become S. chilense, L. cheesmaniae may become S. cheesmaniae or S. galapagense, and L. pimpinellifolium may become S. pimpinellifolium (Solanacea Genome Network (2005) Spooner and Knapp; http://www.sgn.cornell.edu/help/about/solanum_nomenclature.html).

Nucleic acid sequences or fragments comprising ps-2 or DPG genes and alleles may also be defined by their capability to “hybridise” with SEQ ID NO: 1, preferably under moderate, or more preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity. Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

### DETAILED DESCRIPTION OF THE INVENTION

We have recently fine mapped the positional sterility-2 gene, conferring non-dehiscent anthers in tomato (Gorguet et al 2006). We have now isolated the ps-2 gene by positional cloning. Subsequent characterisation of the ps-2 gene revealed that the wild type gene codes for a novel polygalacturonase (PG) and that a single mutation in the coding sequence of gene is responsible for this phenotype of positional sterility due to non-dehiscent anthers. We found that this mutation affects one of the intron splicing recognition site of the gene giving rise to an aberrant mRNA, lacking one of the exons. We have further found that this new PG gene, hereafter designated as Dehiscence PG (DPG) is also expressed in maturing fruits. The jasmonic acid and ethylene play a role in the control of expression of DPG and DPG is involved in the process of fruit ripening.

In a first aspect therefore, the present invention relates to an nucleic acid molecule, preferably, an isolated nucleic acid molecule, comprising a nucleotide sequence encoding a polypeptide with polygalacturonase activity. Polygalacturonase activity and this activity may be determined is defined hereinabove. The nucleotide sequence encoding the polypeptide with polygalacturonase activity may be selected from the group consisting of: (a) a nucleotide sequence encoding an amino acid sequence that has at least 60, 70, 80, 90, 95, 98, 99 or 100% sequence identity with the amino acid sequence of SEQ ID NO. 2; (b) a nucleotide sequence that has at least 55, 60, 70, 80, 90, 95, 98, 99 or 100% sequence identity with the nucleotide sequence of SEQ ID NO. 1; (c) a nucleotide sequence the complementary strand of which hybridises to a nucleotide sequence of (a) or (b); and, (d) a nucleotide sequence the sequence of which differs from the sequence of a nucleotide sequence of (c) due to the degeneracy of the genetic code.

A preferred nucleotide sequence of the invention is from a species within the genus Solanum. More preferably, the nucleotide sequence is from a species within the Solanum Lycopersicum complex, including e.g. S. lycoperisicum, S. chmielewskii, S. habrochaites, S. pimpinellifolium, S. neorickii, and S. pennellii. Alternatively, the nucleotide sequence may be from Solanum melongena. Although a preferred nucleotide sequence of the invention encodes a polypeptide with polygalacturonase activity, expressly included in the invention are alleles, including engineered and mutagenised versions of the nucleotide sequence, that comprise at least one of a substitution, insertion and deletion of one or more nucleotides, and from which no active polygalacturonase can be expressed.

A further preferred nucleic acid molecule of the invention is a molecule that comprises a part of a nucleotide sequence encoding the polypeptide with polygalacturonase activity or an inactive allele thereof. The nucleic acid molecule preferably comprises at least 10, 12, 14, 16, 18, 20, 22, 24, 26, 28, 30, 35, 40 or 50 contiguous nucleotides from nucleotide sequence encoding the polypeptide with polygalacturonase activity or an inactive allele thereof, or more preferably from SEQ ID NO: 1. It is understood that the nucleic acid molecule may comprises more than one stretch of such contiguous nucleotides, which may be directly linked together or separated by other sequences.

In a second aspect the invention relates to a method for detecting, isolating, amplifying and/or analysing a DPG allele in a plant, preferably in a plant of the genus Solanum, more preferably in a plant of the Solanum Lycopersicum complex, or a Solanum melongena plant. The method preferably comprises at least the step of providing a sample comprising nucleic acids of the plant and hybridising the nucleic acids of the plant with a nucleic acid molecule comprising a nucleotide sequence of at least 10 contiguous nucleotides from a nucleotide sequence encoding the polypeptide with polygalacturonase activity, which nucleotides sequence may be selected from the group consisting of: (a) a nucleotide sequence encoding an amino acid sequence that has at least 60, 70, 80, 90, 95, 98, 99 or 100% sequence identity with the amino acid sequence of SEQ ID NO. 2; (b) a nucleotide sequence that has at least 55, 60, 70, 80, 90, 95, 98, 99 or 100% sequence identity with the nucleotide sequence of SEQ ID NO. 1; (c) a nucleotide sequence the complementary strand of which hybridises to a nucleotide sequence of (a) or (b); and, (d) a nucleotide sequence the sequence of which differs from the sequence of a nucleotide sequence of (c) due to the degeneracy of the genetic code. It is understood that the nucleotide sequence of at least 10 contiguous nucleotides may also be taken from an allele of a nucleotide sequence as defined in (a)-(d), including engineered and mutagenised versions of the nucleotide sequence, that comprise at least one of a substitution, insertion and deletion of one or more nucleotides, and from which no active polygalacturonase can be expressed. More preferably the nucleic acid molecule for hybridisation with the sampled nucleic acids of the plant comprises a nucleotide sequence of at least 12, 14, 16, 18, 20, 22, 24, 26, 28, 30, 35, 40 or 50 contiguous nucleotides from a nucleotide sequence as defined above. It is understood that in case the nucleic acid molecule for hybridisation is a single stranded molecule it may also comprise the complement, i.e. opposite strand, of the nucleotide sequence as defined above.

Method for detection, isolation, amplification and/or analysis of specific nucleic acid sequences, such as a DPG allele in a plant, preferably in a plant of the genus Solanum, rely on the sequence specific hybridisation of a nucleic molecule comprising a predetermined sequence, i.e. the nucleic acid molecule for hybridisation, to nucleic acids in the sample to be detected, isolated, amplified and/or analysed. In such method the nucleic acid molecule for hybridisation may thus any nucleic acid molecule capable of hybridising to a DPG allele, preferably a Solanum DPG allele. The molecule may be a probe, e.g. a hybridisation probe or may be a primer to be extended by a polymerase in extension or amplification reaction.

Generally methods for detection, isolation, amplification and/or analysis of specific nucleotide sequences that rely on sequence specific hybridisation of a nucleic molecule comprising a predetermined sequence are well known in the art (see e.g. Sambrook and Russell, 2001, “Molecular Cloning: A Laboratory Manual”, 3^{rd} edition, Cold Spring Harbor Laboratory, Cold Spring Harbor Laboratory Press, New York). More specifically, methods for detection, isolation, amplification and/or analysis of specific DPG allele sequences of the invention may include PCR Amplification (U.S. Pat. Nos. 4,683,195; and 4,683,202; PCR Technology: Principles and Applications for DNA Amplification, ed. H. A. Erlich, Freeman Press, NY, N.Y., 1992), Allele-specific PCR (Gibbs, 1989, Nucleic Acid Res. 17:12427-2448), Allele Specific Oligonucleotide Screening Methods (ASO; Salki et al., 1986, Nature 324:163-166), ligase-mediated allele detection methods such as the Oligonucleotide Ligation Assays (OLA; 1988, Landegren et al., Science 241:107-1080) or the ligation amplification reaction (Wu et al., 1989, Genomics 4:560-569; Barany, 1990, Proc. Nat. Acad. Sci. 88:189-193), Denaturing Gradient Gel Electrophoresis (in Chapter 7 of Erlich, ed., 1992, PCR Technology, Principles and Applications for DNA Amplification, W. H. Freeman and Co., New York), Temperature Gradient Gel Electrophoresis (TGGE), Single-Strand Conformation Polymorphism Analysis (Orita et al., 1989, Proc. Nat. Acad. Sci. 85:2766-2770), differential chemical cleavage of mismatched base pairs (Grompe et al., 1991, Am. J Hum. Genet. 48:212-222), enzymatic cleavage of mismatched base pairs (Nelson et al., 1993, Nature Genetics 4:11-18), systems such as TaqMan™ (Perkin Elmer), Invader Assay, which includes isothermic amplification that relies on a catalytic release of fluorescence (Third Wave Technology at www.twt.com), massive parallel sequencing (e.g. of Roche/454 Life Sciences performed on a GS 20 Genome Sequencer) to produce sequence information for a given locus in thousands of individuals, optionally combined with a 3D pooling strategy (see e.g the Keypoint™ technology at www.keygene.com). and the like.

A preferred method of the invention is a method for detection, isolation, amplification and/or analysis of a ps-2-allele, whereby preferably, the ps-2-allele is an allele that has a C, A or T as last nucleotide of the 3′ end of the fifth exon of the DPG gene, preferably the allele has a C as last nucleotide of the 3′ end of the fifth exon of the DPG gene. This position corresponds to the G at position 3772 of SEQ ID NO:1. Alternatively, molecular markers specific for DPG alleles may be developed. A “molecular marker” is herein understood to refer to a nucleic acid sequence, or a set thereof, that is indicative (directly or indirectly) for the presence or absence of a particular allele, e.g. a DPG allele or a ps-2 allele as herein defined. The presence or absence of the molecular marker can be detected in a wide variety of molecular assays or tests including e.g. Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), and Amplified Fragment Length Polymorphisms (AFLPs). A molecular marker specific for a DPG allele is herein understood as a marker that is present within the genome of the plant, preferably the Solanum plant no more than 100, 50, 20, 10, 5 or 2 kb from the a nucleotide sequence encoding the polypeptide with polygalacturonase activity as defined hereinabove or a part thereof. Such molecular markers may be used for detection of specific DPG alleles, including ps-2-alleles.

In a third aspect the invention relates to the use of a nucleic acid molecule for hybridisation as defined in herein above in marker-assisted breeding. Preferably, the marker-assisted breeding comprises the detection of a ps-2-allele. A ps-2-allele is herein understood as a DPG allele of a plant, preferably a plant of the genus Solanum, more preferably in a plant of the Solanum Lycopersicum complex or in a Solanum melongena plant, that, when homozygous produces a positional sterility phenotype that is due to non-dehiscent anthers. A ps-2-allele will usually be an allele from which in a plant that is homozygous for the allele insufficient DPG activity is expressed to prevent positional sterility and non-dehiscent anthers. More preferably, the marker-assisted breeding comprises the detection of a ps-2-allele that has a C, A or T as last nucleotide of the 3′ end of the fifth exon of the DPG gene, of which a C as last nucleotide of the 3′ end of the fifth exon of the DPG gene is most preferred.

In a fourth aspect the invention relates to a method for producing a plant with non-dehiscent anthers. Preferably the plant is a Solanum plant, more preferably a plant of the Solanum Lycopersicum complex, or a Solanum melongena plant. The plant preferably has a phenotype of positional sterility. The method preferably comprises the steps of: (a) crossing a first plant with a second plant that is homozygous for a ps-2-allele; (b) backcrossing the F1 generation and further generations for at least one (preferably at least two) generation with the first plant as recurrent parent; and, (c) selfing the furthest backcrossed generation obtained in b) for at least one (preferably at least two) generations; wherein a molecular marker is used in at least one of steps b) and c) to select for a plant that is homozygous for the ps-2-allele. Preferably in the method the molecular marker is a marker specific for a DPG allele as herein defined, more preferably the marker is specific for a ps-2-allele as herein defined. Most preferably, the molecular marker is or detects a C, A or T as last nucleotide of the 3′ end of the fifth exon of the DPG gene, of which a C as last nucleotide of the 3′ end of the fifth exon of the DPG gene is most preferred.

In a fifth aspect the invention relates to methods for producing a plant with a mutation or genetic modification in a DPG-allele. Preferably the plant is a Solanum plant, more preferably a plant of the Solanum Lycopersicum complex, or a Solanum melongena plant. The mutation or genetic modification may be introduced in the locus of a DPG encoding nucleotide sequence/gene using the technique of TILLING (Targeted Induced Local Lesions IN Genomes). Methods for TILLING are well known in the art (McCallum et al., 2000 Nat Biotechnol. 18(4):455-7; reviewed by Stemple, 2004, Nat Rev Genet. 5(2):145-50.). The TILLING mutagenesis technology is useful to generate and/or identify, and to eventually isolate mutagenised variants of a DPG genes, including ps-2-alleles. TILLING also allows selection of plants carrying such mutant variants. TILLING combines high-density mutagenesis with high-throughput screening methods. The steps typically followed in TILLING are: (a) mutagenesis, e.g. by Ethyl methanesulfonate (EMS) or N-ethyl-N-nitrosourea (ENU); (b) growth of plant from mutagenised seed, and optionally, backcrossing the plants for at least one generation; (c) preparation of DNA from tissue samples of the plants and, optionally, pooling of DNAs from individuals; (d) PCR amplification of a region of interest, i.e. the DPG gene, e.g using primers as hereinabove defined; (e) detection of mutant PCR products (f) identification of the mutant individual and (h) optionally, sequencing of the mutant PCR. Initially in the method detection of mutant PCR products in step (e) and optionally (f) was performed by denaturation and annealing to allow formation of heteroduplexes and subsequent detection of heteroduplexes by DHPLC (Denaturing High Performance Liquid Chromatography) (McCallum et al., 2000, supra). The method was made more high throughput by using the restriction enzyme Cel-I combined with a gel based system to identify mutations (Colbert et al., 2001, Plant Physiol. 126(2):480-4). Other methods for detection or identification of single base mutations in the target DPG gene that may be used in methods of TILLING include e.g. resequencing DNA as has been described (see e.g. Slade et al., 2005, Nat Biotechnol. 23(1):75-81) or massive parallel sequencing (e.g. of Roche/454 Life Sciences performed on a GS 20 Genome Sequencer) to produce sequence information for a given locus in thousands of individuals, optionally combined with a 3D pooling strategy (see e.g the Keypoint™ technology at www.keygene.com). Mutagenesis may be performed by radiation or with a chemical mutagen such as EMS (Lightner and Caspar, 1998, “Seed mutagenesis of Arabidopsis”, In: Methods in Molecular Biology: Arabidopsis Protocols (eds. J. M. Martinez-Zapater and J. Salinas), pp. 91-103. Totowa, N.J.: Humana Press) or ENU (Draper et al., 2004, Methods Cell Biol. 2004; 77:91-112). A preferred method for producing a plant with a mutation in a DPG-allele preferably at least includes the steps of: (a) mutagenising seeds of a plant; (b) growing plants of the mutagenised seeds obtained in a); (c) optionally, backcrossing the plants obtained in b) for at least one generation; and (d) screening plants obtained in b) or c) for the presence of a mutation in a DPG-allele. Preferably the plant is a Solanum plant, more preferably a plant of the Solanum Lycopersicum complex, or a Solanum melongena plant. Preferably the mutation in the DPG-allele cause the allele to be a ps-2-allele. More preferably, the ps-2-allele is an allele that has a C, A or T as last nucleotide of the 3′ end of the fifth exon of the DPG gene, most preferably the allele has a C as last nucleotide of the 3′ end of the fifth exon of the DPG gene.

In a sixth aspect the invention relates to methods for producing a transgenic plant, preferably a Solanum plant with non-dehiscent anthers. Transgenic plants with non-dehiscent anthers may be obtained in various manners including e.g. silencing of the DPG gene by introduction of antisense, sense suppression or RNAi (RNA interference) nucleic acid constructs into the plants, or by knock-out of the DPG gene by homologous recombination. The method thus comprises at least the step of transforming a plant cell with a nucleic acid construct comprising a nucleotide sequence at least a fragment of a nucleotide sequence encoding a DPG as defined hereinabove, wherein presence of the nucleic acid construct in cell of the plant reduces expression of DPG activity to a level that effects positional sterility and non-dehiscent anthers. Plants with non-dehiscent anthers may derived from transformed plant cells or from plants comprising such transformed cells by methods known to the skilled person per se.

Thus in a preferred embodiment the nucleotide sequence in the nucleic acid construct is operably linked to a promoter for expression in a plant cell, e.g. a Solanum cell, and expression of the nucleotide sequence reduces expression of DPG activity by RNA interference. Methods and nucleic acid constructs for gene silencing are described in US20070130653, US20070074311 and references cited therein. The invention thus relates to a nucleic acid molecule that is a construct comprising at least a fragment of a nucleotide sequence encoding a DPG as defined hereinabove is operably linked to a promoter for expression in a plant cell. In the nucleic acid construct the fragment preferably comprises a sequence of 30, 60, 100, 200 or 500 contiguous nucleotides from a nucleotide sequence having at least 60, 70, 80, 90, 95 or 100% sequence identity to a nucleotide sequence encoding a DPG as defined hereinabove, or a complement thereof, more preferably the nucleotide sequence encoding a TDPG is SEQ ID NO:1.

In another preferred embodiment, the transgenic plants with non-dehiscent anthers may be obtained by knock-out of the DPG gene by homologous recombination. In this method the nucleic acid construct preferably is a construct for homologous recombination and the nucleotide sequence preferably comprises a mutation that reduces expression of DPG activity to a level that effects positional sterility and non-dehiscent anthers. This mutation may be a mutation in the broadest sense, including e.g. complete deletion of coding and/or promoter sequence or insertion of selectable marker or other sequences to inactivate the DPG gene. Homologous recombination allows introduction in a genome of a selected nucleic acid at a defined selected position. Methods for performing homologous recombination in plants have been described not only for model plants (Offring a et al., 1990 EMBO J. 9(10):3077-84) but also for crop plants, for example rice (Iida and Terada, Curr Opin Biotechnol. 2004 April; 15(2):132-8). The nucleic acid to be targeted is thus preferably an deficient DPG allele, e.g. a ps-2-allele, used to replace the endogenous DPG gene.

Plants of the invention with the non-dehiscent anthers and/or positional sterile phenotypes have the advantage that they allow more cost-effective production of hybrid plants by avoiding or reducing self-fertilisation. Production of hybrids may involve the use of male sterilities such as CMS, genetic sterility or positional sterility. Positional sterility is preferred because one the one hand it reduces or prevents undesired self-fertilisation whereas on the other hand if so required it does allow self-fertilisation by (manual) opening or damaging of the anthers. The DPG alleles of the invention may thus be used as a tool to prevent inbreeding during the creation of hybrids that may or may not have a positional sterile phenotype due to non-dehiscent anthers (depending on the phenotype of the male parent). In addition the non-dehiscent anthers and/or positional sterile phenotypes may advantageously be applied in the production of seedless fruit in avoiding or reducing (self)-fertilisation and thereby improving the seedless phenotype.

Although the invention has been exemplified by means of tomato plants, it is understood that the invention includes DPG nucleic acids, ps-2 alleles, and methods for producing (transgenic) plants of all commercially important crops including e.g. Asteraceae (including the food crops lettuce, chicory, globe artichoke, sunflower, yacón, safflower) Cucurbitaceae (commonly known as gourds or cucurbits and includes crops like cucumbers, squashes (including pumpkins), luffas, melons and watermelons), Brassica (including swedes, turnips, kohlrabi, cabbages, brussels sprouts, cauliflower, broccoli, mustard seed and oilseed rape), Leguminous Crops (including dry beans, dry broad beans, dry peas, chickpea, garbanzo, bengal gram dry cowpea, black-eyed pea, blackeye bean, pigeon pea, toor, cajan pea, congo bean, lentil, bambara groundnut, earth pea vetch, common vetch Lupins, soybean, peanut) Chenopodiaceae (spinach). Liliaceae (unions, leek), Apiaceae (carrots), Grain crops (rice, barley, wheat, oats, corn), Solanaceae (including tomato, pepper, eggplant).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DESCRIPTION OF THE FIGURES

FIG. 1: Microscopic observation of anthers of ps-2ABL and Wild-Type (Moneymaker).

a: Cross section of anthers at late flower bud (FB), early pre-anthesis (PA) stained in toluidine blue. Arrows refer to the crystals that can be seen in the septum cells of WT and ps-2ABL. s: septum; st: stomium; p: pollen.
b: Cross section of anthers at anthesis stained in toluidine blue. Arrows indicate the opening of the anther in WT and the stomium that remains closed in ps-2ABL.
c: longitudinal section of anther cones at anthesis observed by SEM. Arrows indicate the longitudinal opening if the anther in WT and the unopened anthers in ps-2ABL.

FIG. 2: Cloning of the ps-2 gene, from genetic map to candidate gene

a: Genetic map developed in a recombinant F2 population (ps-2ABL×S. pimpinellifolium; Gorguet et al, 2006). In white are the numbers of recombinant plants between each marker.
b: Physical map: Arrows in dashed represent the computational anchoring of BACs. Arrows in full line represent the anchoring of BACs by molecular markers. (S: SP6; T: T7).
c: Genetic positions of the ORFs on BAC clone 143M15.
d: Structure of candidate gene ORF4. Coloured cylinders with roman numbers represent exons. Positions of the putative TATA box and PolyA are indicated by an inverted blue triangle at the beginning of the sequence and a green rhombus at the end of the sequence.

FIG. 3: RT-PCR performed on RNA from anther cones at post-anthesis from ps-2ABL and cv. Moneymaker.

FIG. 4: Intron splicing between exon IV and VI of ORF4, in Moneymaker (WT) and in ps-2ABL in anthers.

FIG. 5: Phylogenetic tree for 20 angiosperm and 1 gymnosperm PG aa sequences. The phylogram is the result of a 50% Majority-rule consensus of 3296 trees (using tree weights). The PG sequences segregate into two major clades identified as the well characterized clades A and B. the candidate protein (TDPG) is part of clade B.

FIG. 6: Alignment of Pfam GH28 domain of TFPG, ORF4 and ps-2 (mutant of ORF4). Colour code is only effective for the comparison between TFPG and ORF4: In reverse colour are the identical amino acids. In grey are the conserved substitutions. In bold underlined are the four conserved domains of polygalacturonase as defined by Rao et al (1996).

FIG. 7: Tissue specific expression of ORF4. cDNA of Moneymaker from diverse tissues were subjected to PCR amplification using primers dedicated to quantitative PCR analysis. Genomic DNA of Moneymaker was used as control for contamination 1: Leaf abscission zone; 2: Flower abscission zone; 3: Anthers at anthesis; 4: Fruit at mature stage.

FIG. 8: Simplified model for the hormonal control of anther dehiscence and fruit maturation in tomato in which TDPG is taking part.

### EXAMPLES

### 1. Example 1

### 1.1 Materials and Methods

### 1.1.1 Plant Material

The F₂ recombinant sub-population developed from the cross between ps-2ABL (a true Advanced Breeding Line S. lycopersicum, homozygous for the ps-2 mutation) and S. pimpinellifolium, was used for genetic mapping. This population, segregating for ps-2, is composed of 146 F₂ plants recombinants in the ps-2 locus region (Gorguet et al, 2006).

Another 176 ABLs, eight of them being ps-2/ps-2, were used to test the association between the identified SNP and the ps-2 locus.

Anthers of ps-2ABL and cv. Moneymaker were used for microscopic observations.

### 1.1.2 Microscopy

Plant material was fixed for 24 h at 4° C. in 0.1 M phosphate buffer, pH 7.0, containing 4% paraformaldehyde. Samples for scanning electron microscopy were processed as described in Domelas et al. (2000), and digital images were obtained using an Orion Framegrabber (Matrox Electronic Systems, Unterhaching, Germany). Samples for light microscopy were embedded in Technovit 7100 (Hereaus Kulzer, Wehrheim, Germany), stained with toluidine blue, and mounted in Euparal (Chroma-Gesellschaft, Kongen, Germany).

1.1.3 BAC Library Screening and contig Construction

We used the tomato HindIII BAC library constructed from genomic DNA of the cultivar accession Heinz 1706. The Heinz library is a 15 genomes equivalent with an average insert size of 114.5 kb (Budiman et al., 2000). Screening of the BAC library was performed by PCR amplification, first on plate pools and then on individuals. Plasmid DNA of the positive BAC clones was then isolated and used for further analysis.

BAC ends sequences of the positive BAC clones were obtained from the SGN database (Mueller et al. 2005). Conversion of the BAC ends sequences into CAPS or dCAPS markers was performed as described by Gorguet et al. (2006). Details of the PCR markers derived from BAC ends sequences are presented in Table 1.

TABLE 1

PCR markers used in the genetic linkage maps

| | | | | |
|---|---|---|---|---|
| Marker | | | | Restriction |
| name | Use | Primer sequence | Size (bp) | enzyme |

Markers developed from BAC ends sequences

| | | | | |
|---|---|---|---|---|
| 67F23-T | CAPS | Fw: CTACTCTTCCGCCATAACTG | 599 | HincII |
| | | Rv: GATCCAAACGAACAAAAGTCA | | |
| 67F23-S | CAPS | Fw: TCATTCCGTTGCTGAATGAGA | 413 | DraI |
| | | Rv: ATAACTTATATCACTCCCAATCA | | |
| 69C22-T | dCAPS | Fw: TCTTTCGATATTTTTCAGAACTAA | 200 | DdeI |
| | | Rv: TGAGATGTTTGCAATAACATTCT | | |
| 143M15-S | CAPS | Fw: CATCGAAGTAACAGAGATATTA | 369 | MwoI |
| | | Rv: CCATAGGGATTATGATGTGTA | | |
| 114C15-T | CAPS | Fw: GCACTGAAGAATGGATAGACTC | 457 | MnlI |
| | | Rv: GGAATTGACCAAAAAGGATAGC | | |
| 118A17-T | CAPS | Fw: GGCATGGTGAAGTCCACATT | 739 | HaeIII |
| | | Rv: GTGTCACAGGTTTGGTTCAT | | |
| 15N23-T | CAPS | Fw: GGCAGATATCTGCAATACGT | 576 | TaqI |
| | | Rv: ATCATGAACAGCAAAACAACCA | | |

Markers developed from BAC 143M15 sequence

| | | | | |
|---|---|---|---|---|
| ORF1 | CAPS | Fw: CTGTATCTATGACGAGGAGA | 625 | HaeIII |
| | | Rv: GATCCTGAAGCTGAAGCTT | | |
| ORF2 | CAPS | Fw: AATATTTTCAACTTTCAAATCTCTT | 206 | MnlI |
| | | Rv: ACGAAGGCATGATTGTCGTTA | | |
| ORF3 | CAPS | Fw: GTTGAACTTATACCACTAGGA | 937 | NdeI |
| | | Rv: GTGCGGTCTCATCAACTCAA | | |
| ORF4 | dCAPS | Fw: GAACACTTAGGTTAAAATATAGC | 217 | AluI |
| | | Rv: CCTACTATCCTTCTTGTAATCT | | |
| ORF5 | CAPS | Fw: CTTAAAGGCACACTTAGATTCA | 962 | HpyCH4IV |
| | | Rv: CTGAGAATTCTCTTGACTGCA | | |
| ORF4(1) | SCAR | Fw: GCTTTATTCATAGTAAATTCTGT | 805/885 | |
| | | Rv: TCAGACAAATCATCGTATATTGA | | |
| ORF4(2) | SCAR | Fw: TCCATTTGTAGTTTCATAAAGC | 465/515 | |
| | | Rv: CCAAGCGGATAATTAATGTCA | | |

Marker developed for ps-2ABL allele identification within
S.lycopersicum

| | | | | |
|---|---|---|---|---|
| ps-2 marker | CAPS | Fw: CAAATTGGATGAGAGTTTTGAA | 695 | HpyCH4IV |
| | | Rv: CATTTTACAAGTGTAACAACTTG | | |

Fingerprinting patterns of individual BAC clones were generated essentially as described by Brugmans et al (2006), using the Hind III/TaqI enzyme combination.

### 1.1.4 BAC DNA Sequencing and Analysis

The size of BAC clone 143M15 was estimated by pulse field gel electrophoresis. BAC clone 143M15 was sequenced via the shotgun-sequencing method by Greenomics (The Netherlands). PCR markers derived from the candidate genes identified on the BAC sequence were developed as described by Gorguet et al. (2006). To identify putative genes, the final BAC DNA sequence was scanned against the tomato Unigene database from SGN (Mueller et al, 2005) and the Arabidopsis gene models database from TAIR (http://www.Arabidopsis.org; Huala et al., 2001), using the TBLASTX interface of SGN (Mueller et al., 2005), with a significance threshold of 1E-10. PCR markers based on putative ORF sequences were developed and screened on the recombinant population as described by Gorguet et al. (2006). Details of these PCR markers are given in Table 1.

### 1.1.5 Candidate Gene Analysis and Phylogenetic Analysis

The complete genomic DNA sequence of ORF4, as well as the up-stream and down-stream sequences containing respectively the promoter and the gene terminator was amplified and sequenced in Moneymaker and the ps-2ABL using several successive overlapping primer pairs giving products of around 900 bp. The resulting DNA sequences of Moneymaker and ps-2ABL were assembled with DNAStar. Softberry gene finding software was used to identify the putative exons and introns of the candidate gene.

Tomato polygalacturonase protein sequences with known tissue expression, as well as the best hits of a protein BLAST search with the candidate protein, were selected to conduct a phylogenetic analysis. Only the Pfam Glycosyl Hydrolase 28 domains, of the selected protein sequences, were used for the analysis. The Pfam GH28 domain of each protein sequence was identified with the protein Blast interface of NCBI. Selected amino acid sequences of Pfam GH28 domains were aligned using ClustalW multiple sequence alignment software (Higgins et al, 1994).

PAUP software package version 4 (Swofford 2002) was used to construct a 50% majority-rule consensus phylogenetic tree using maximum parsimony (1000 bootstrap replicates and 250 addition sequences replicates). Cedar PG protein sequence was used and defined as outgroup.

The amino acid sequences and their protein identification numbers were: Kiwi fruit (AAC14453; Atkinson and Gardner, 1993), grape berry fruit (AAK81876; Nunan et al, 2001), soybean pods (AAL30418; Christiansen et al, 2002), peach fruit (CAA54448; Lester et al, 1994), apple fruit (AAA74452; Atkinson, 1994), pear fruit (BAC22688; Hiwasa et al, 2003), Arabidopsis dehiscence zone ADPG1 (CAA05525; Sander et al, 2001), oilseed rape dehiscence zones RDPG1 (CAA65072; Petersen et al, 1996), oilseed rape pod (CAA90272; Jenkins et al, unpublished), turnip silique valve desiccation (CAD21651; Rodriguez-Gacio et al, 2004), Bell pepper fruit (BAE47457; Ogasawara S and Nakajima T, unpublished), tomato fruit TFPG (CAA32235; Bird et al, 1988), tomato pistil (AAC70951; Hong and Tucker, 2000), tomato abscission zones TAPG1, 2, 4, 5 (AAC28903, AAB09575, AAB09576, AAC28906; Hong and Tucker, 1998), tomato wound leaf (AAD17250; Bergey et al, 1999) and tomato seed (AAF61444; Sitrit et al, 1999).

1.1.6 Total RNA Isolation, cDNA Synthesis and Quantitative PCR Analysis:

Total RNA was isolated using the RNeasy Plant Mini Kit (Qiagen, Hilden, Germany). Between 50 and 100 mg of each tissue (anthers, fruit, flower abscission zone and leaf abscission zone) was used per RNA isolation reaction. Only 1 μg of total RNA was used per sample for the synthesis of cDNA, after DNase I treatment (Boehringer Manheim). First strand cDNA template was synthesized using random hexamers as primers and Multiscribe™ Reverse Transcriptase (Applied Biosystems). The quasi complete coding sequence of ORF4 was amplified to study the intron splicing using the forward primer: TAGCTCCAAAGCTATCCACAT, located on the first exon, starting 47 nucleotides down-stream the start codon and the reverse primer: TGGAGAATGTGAAATTGTTAGG, located on the last exon, stopping 100 nucleotides up-stream the stop codon. Quasi-complete CDS of ORF4 was amplified with standard PCR reaction (55° C. annealing temperature and 35 cycles)

Real-time experiments were conducted in an iCycler MyiQ detection system (Bio-Rad), using the SYBR green PCR master mix kit (Applied Biosystems). Primer sequences were: forward primer 5′-TTTTGCCATTGCCATTGATA-3′, reverse primer 5′-TGTGGTGTCCCAGAACAAGA-3′ (ORF4); and forward primer 5′-ACCACTTTCCGATCTCCTCTC-3′, reverse primer 5′-ACCAGCAAATCCAGCCTTCAC-3′ (β-actin). Relative quantification of the ORF4 transcript level was calculated with the internal β-actin control by applying the 2^{-ΔCT} formula. Purity of the PCR products was verified with the melting curves. The reactions were done in duplo. PCR controls were performed in absence of added reverse transcriptase to ensure RNA samples were free of DNA contamination.

### 2. Results

### 2.1 Microscopic Observation of the ps-2 Phenotype

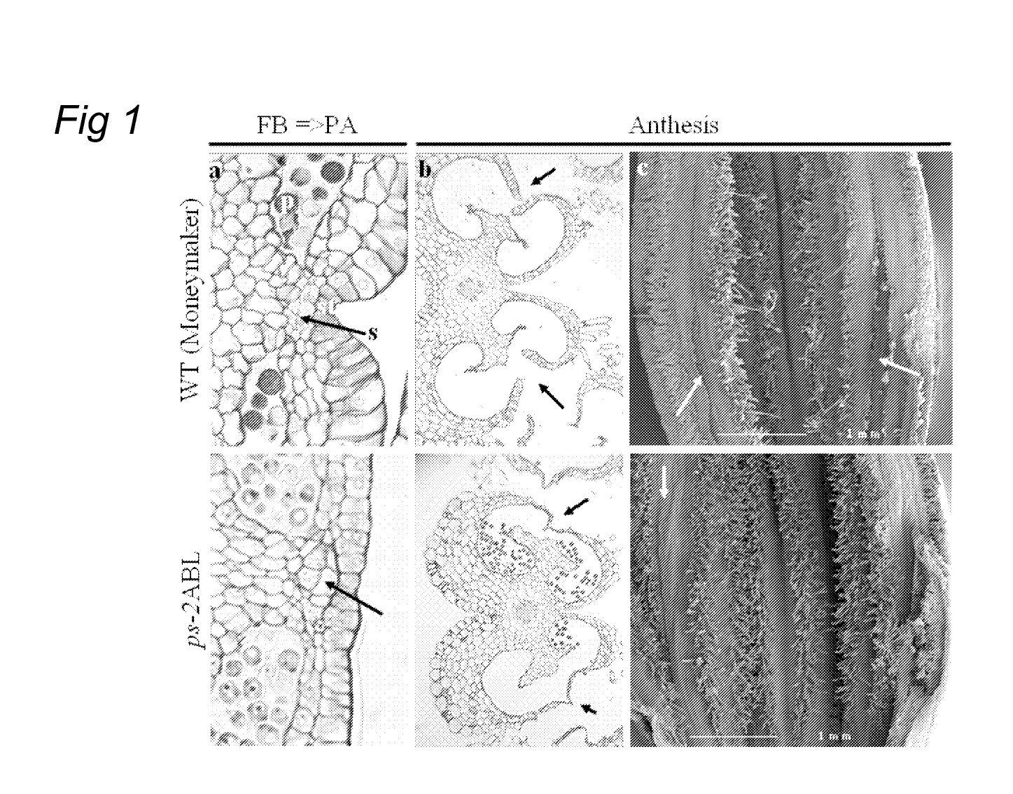

Transverse section of anthers of wild-type (Moneymaker) and mutant (ps-2ABL) were prepared and stained with toluidine blue to identify the stage at which anther development/dehiscence in the mutant is blocked. Longitudinal sections of anther cones at anthesis were prepared for electron microscopy. No difference in developmental stages was visible until anthesis. At late Flower Bud, crystals were observed in the septum cells of mutant and wild-type, and pollen development appeared normal (FIG. 1a). Breakage of the septum did occur in the mutant at similar stage than the wild type. However at anthesis the mutant stomium did not degenerate and the pollen remained in the anthers (FIG. 1-b,c). In addition, endothecial thickening did not occur in the mutant and therefore the epidermal cells lacked rigidity at anthesis stage in order to create a breaking force on the stomium.

### 2.2 Physical Mapping and Candidate Gene Identification

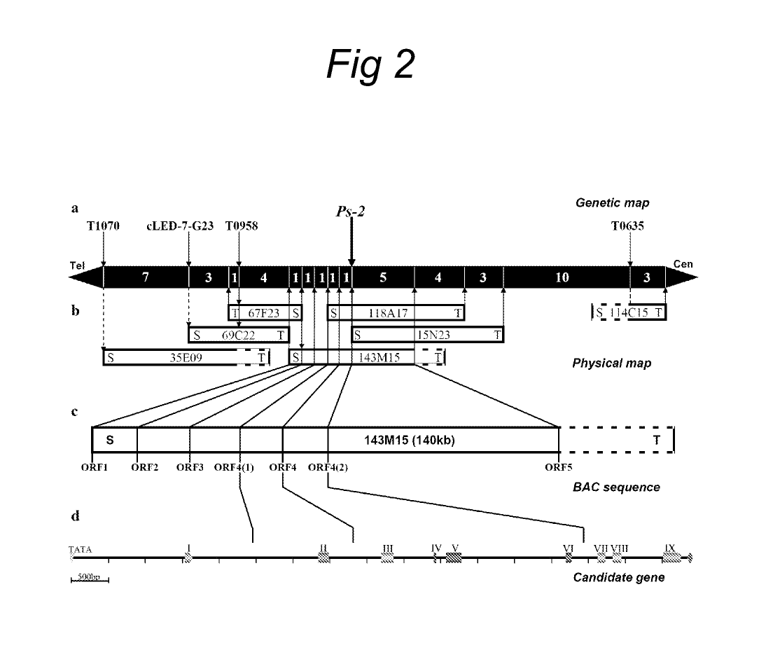

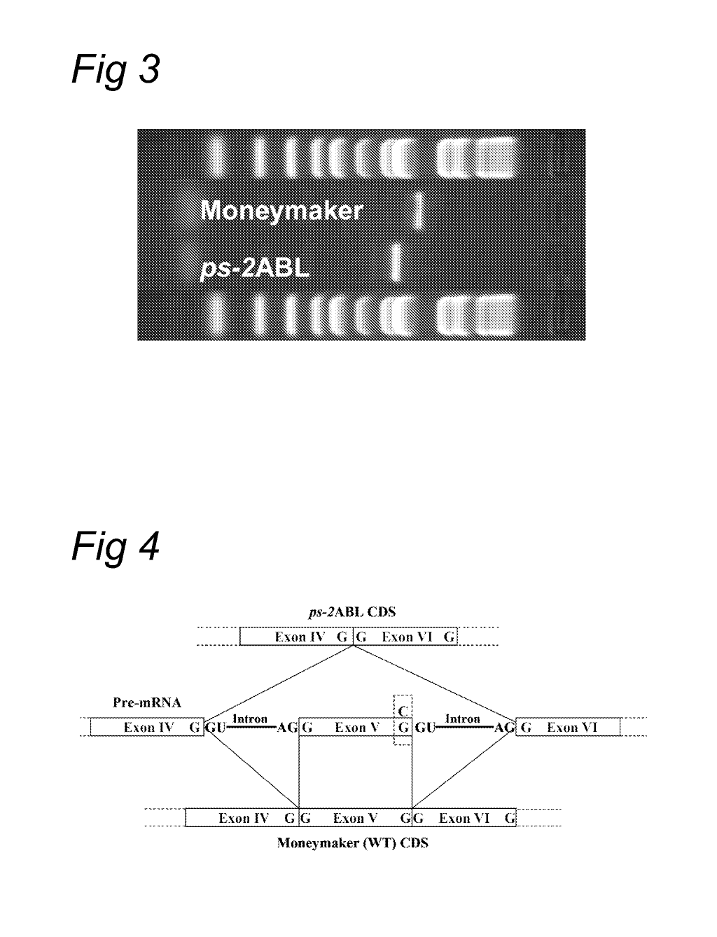

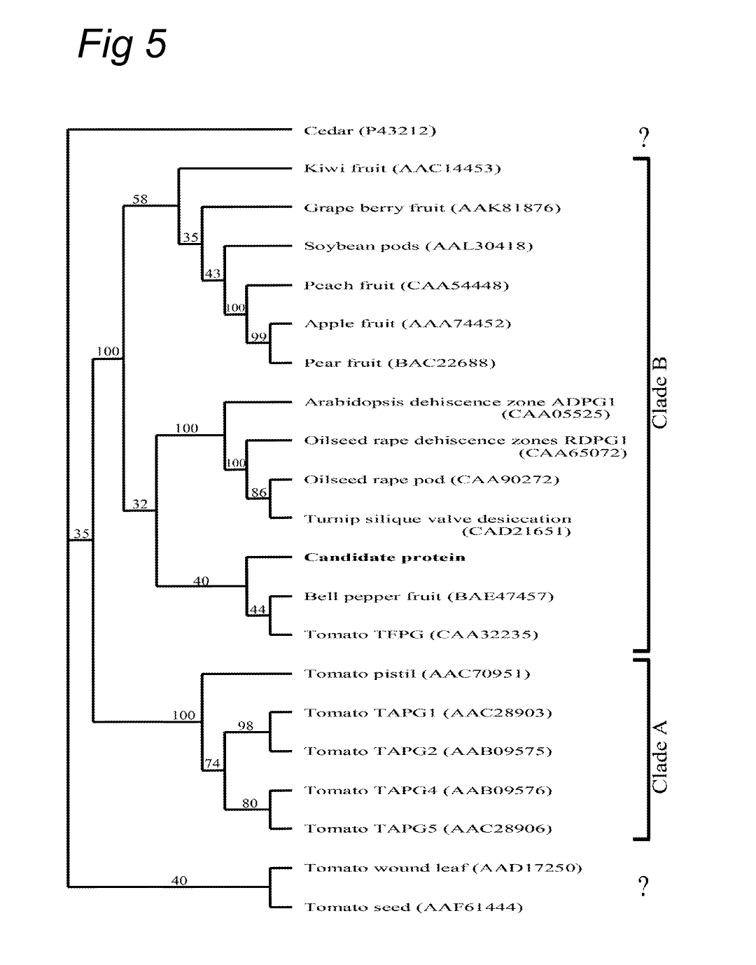

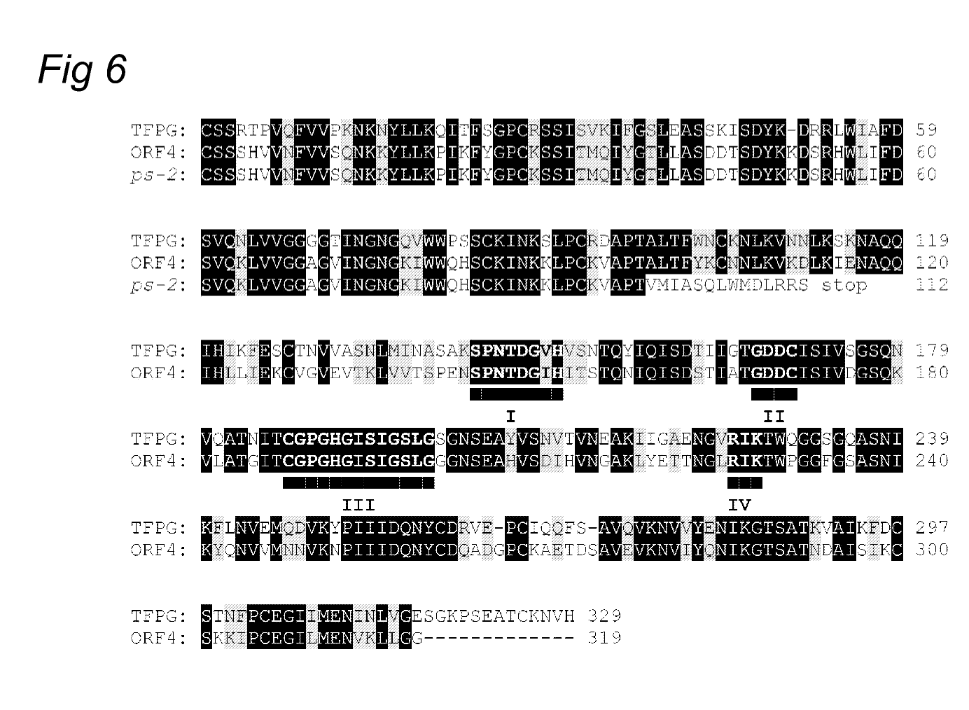

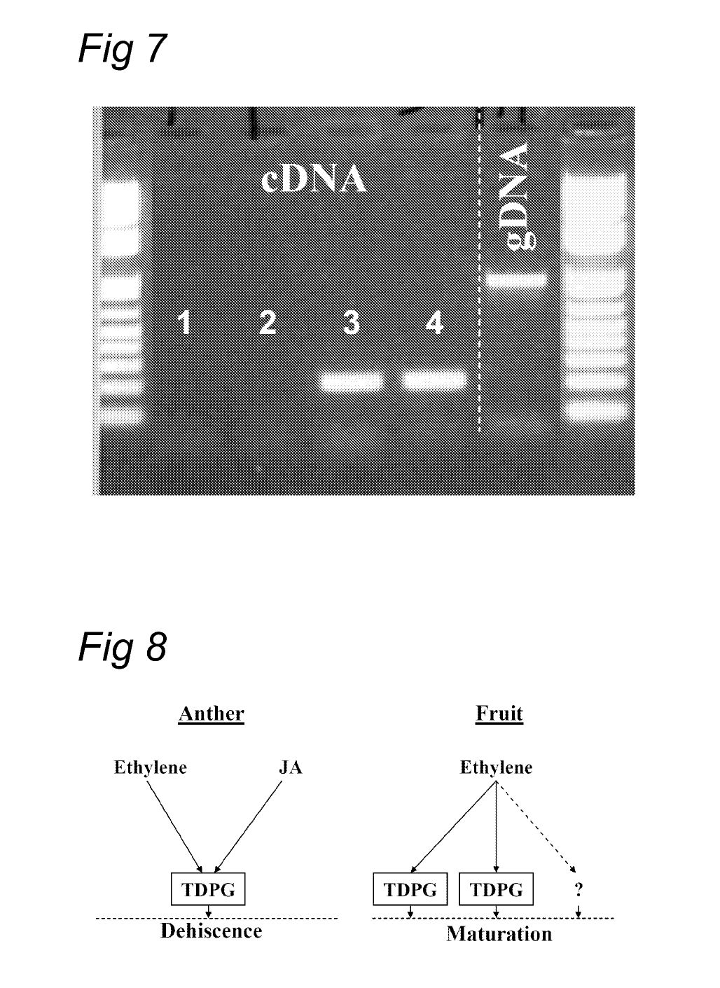

We have previously mapped the ps-2 locus to an interval of 1.65 cM defined by COS derived CAPS markers T0958 and T0635 on the short arm of Chromosome 4 (Gorguet et al, 2006). A physical map for the ps-2 locus region was built using the Heinz BAC library. The library was screened by PCR amplification with the closest markers relative to the ps-2 locus and by computational means using the sequences of those markers. Positive BACs were then anchored to the genetic map by converting BAC ends into PCR markers (FIG. 2) and by screening these markers in the recombinant population. BAC fingerprints were also compared to evaluate the overlapping of BACs from the same contig and to verify whether BACs were part of the same contig. The closest BAC end relative to the ps-2 locus was then used for a second round of screening. Eventually, the entire contig spanned 1.70 cM (32 recombinants) from COS derived CAPS marker T1070 to BAC end 15N23-T (FIG. 2). BAC 143M15 spanned the ps-2 locus and was therefore sequenced.

In order to identify the genes present in BAC clone 143M15, the BAC DNA sequence was scanned against the Tomato SGN Unigene database, using a BLASTN interface, and against the Arabidopsis gene models database, using a TBLASTX interface. Two tomato coding sequences and five Arabidopsis genes matched the BAC clone sequence (two of them matching the two tomato coding sequences; Table 2). The five candidate genes were named ORF1 to ORF5. In addition, six genes of the retrotransposon family were also identified but were not taken into account in the further study. The positions of the five corresponding Arabidopsis genes were not contiguous in the Arabidopsis genome which highlighted the absence of synteny between the two species for that region. Moreover none of them was homolog to one of the functional male sterility genes identified in Arabidopsis (listed in Gorguet et al., 2006).

TABLE 2
Identification of candidate gene based on Arabidopsis gene models

| | Tomato | Arabidopsis | | | | | |
|---|---|---|---|---|---|---|---|
| | Unigenes | Accession | | | AGI coordinates | TBLASTX | |
| Gene | Acc. no. | no. | Gene function | Chr. | (bases) | E value | Score |
| ORF1 | U323899 | AF014399 | magnesium-chelatase | 1 | 2696415-2700961 | 0 | 214 |
| ORF2 | | NM112785 | transcriptional factor B3 family | 3 | 6548875-6551847 | 3E−21 | 105 |
| ORF3 | U317249 | AF326883 | remorin family protein | 2 | 17477944-17480014 | 9E−37 | 110 |
| ORF4 | | NM111676 | polygalacturonase | 3 | 2541012-2543438 | 1E−79 | 108 |
| ORF5 | | AB017502 | glycosyl hydrolase family 3 | 5 | 7107378-7111311 | 0 | 276 |

In order to localize the candidate genes in the high resolution map, we converted the putative genes sequences (or the sequences nearby) into PCR markers and mapped them in the recombinant population. Every candidate genes mapped at different positions in the high resolution linkage map and therefore we could easily identify the likeliest candidate for ps-2 based on their positions in the genetic map. ORF4, a putative polygalacturonase gene, mapped the closest to the ps-2 locus (FIG. 2). Subsequently the putative introns and exons were identified using the FGENSH software of Softberry. The candidate gene ORF4 is composed of nine exons and eight introns, covering a genomic distance of 6716 nucleotides from putative start to stop codon, for a coding sequence of 1179 nucleotides. The SNP used to develop the PCR marker ORF4 was located in the second putative intron. We developed and mapped two extra PCR markers, one based on a deletion of 76 bp in the first intron in the S. pimpinellifolium allele [ORF4(1)] and one based on an insertion of 38 bp in the sixth intron in the S. pimpinellifolium allele [ORF4(2)]. The three PCR markers, ORF4(1), ORF4 and ORF4(2) mapped at an interval of one recombinant between each other, indicating that at least two recombinations had occurred within the candidate gene ORF4 in the recombinant population (FIG. 2). ORF4(2) co-segregated with the ps-2 locus on the high resolution map.

### 2.3 Mutation in ORF4 and Molecular Marker Development

To consolidate the hypothesis that ORF4 corresponds to the ps-2 gene, we searched for sequence alterations in the ps-2ABL allele. The entire 9 kb, from the putative promoter to the putative terminator of ORF4 was sequenced on cv. Moneymaker and ps-2ABL. One single mutation was identified on the last nucleotide of the fifth putative exon of ORF4, in which the nucleotide Guanine was replaced by Cytosine. To test the association between the identified SNP in ORF4 and the ps-2 trait, we developed a molecular marker based on that SNP, in such a way that the ps-2ABL allele and the wild type allele in S. lycopersicum could easily be differentiated on gel. This marker was tested on a set of 176 ABLs among which eight were ps-2/ps-2. These seven functionally male sterile plants showed the same marker pattern, distinct from the other ABLs, which confirm that the SNP is present in the other ps-2 lines tested. This marker can now easily be used for molecular assisted introduction of the ps-2 trait into modern tomato lines.

### 2.4 Alternative Intron Splicing

Because the sequence mutation in the ps-2ABL allele of ORF4 is located in one of the intron recognition splice sites, we hypothesized that this mutation could affect the pre-mRNA splicing of the gene. In order to verify this hypothesis we designed primers to amplify the quasi full-length cDNA clone of ORF4 (1032 nt out of 1179 nt): The forward primer was designed on the first exon of ORF4 and the reverse primer on the last exon (Table 3). RT-PCR was performed on cDNA made from RNA of anther cones at post-anthesis from ps-2ABL and cv. Moneymaker. The observed amplified product for cv. Moneymaker was of the expected size (1032 bp), which was confirmed by sequencing. The amplified product of ps-2ABL was significantly smaller than the Moneymaker product which suggested an alteration in the introns splicing (FIG. 3). The sequencing of the fragment amplified on cDNA of ps-2ABL showed that the fifth exon, on which the SNP is present, was skipped in the cDNA sequence. This exon was removed together with the two flanking introns during the pre-mRNA maturation process (FIG. 4). The wild type 5′ sequence of this exon-intron splice junction is CAG/GTATCG, which is identical to one of the splice junction sequence identified in Solanum tuberosum (Brown, 1986). The mutation found in the ps-2ABL allele induces the following sequence: CAC/GTACG, which is not present in the list of intron splicing recognition sites. The absence of the fifth exon in the mature mRNA represents a deletion of 208 nucleotides, which induces a frame-shift on the remaining coding sequence down-stream. This frame-shift causes a premature termination of translation after 14aa due to a newly framed stop codon. The complete putative mutated protein is therefore 154 aa long in comparison to 392 aa for the wild type protein, and is likely to be non-functional.

### 2.5 ORF4 Sequence Analysis

A BLAST search with the putative candidate protein sequence of ORF4, in the protein database of NCBI, resulted in a list of PG proteins from several plants. The identified proteins have functions in fruit ripening and siliques/pods dehiscence. Among them, the ADPG1 protein had been found to be expressed in the dehiscence zone of siliques of Arabidopsis as well as the dehiscence zone of anthers (Sander et al, 2001). Amino acids of the Pfam GH28 domain of the best BLAST hits were aligned together with the sequences of the candidate protein and the already known tomato PG proteins with identified functions, and one gymnosperm PG (cedar). A phylogenetic analysis was performed on the final alignment in order to place the candidate protein in one of the referenced PG clade. ORF4 was identified as a PG of clade B (FIG. 5). Clade B is composed of all the cloned genes that encode PG expressed in fruit and dehiscence zone, as previously characterized by Hadfields and Bennett (1998). This was also observed in the present phylogenetic tree. TFPG, the only tomato PG known to be expressed in fruits, was also part of the same clade. Alignment of Pfam GH28 domains of ORF4 and TFPG is presented in FIG. 6. ORF4 and TFPG have a similarity of 59% on the entire protein sequence.

The putative derived protein of ORF4 contains the four conserved domain characteristic of PG proteins, as presented by Rao et al (1996; FIG. 6). The first conserved domain is located on the fifth exon, which is skipped in the mutant protein and the three others domains are located further on the sequence and therefore not in framed in the mutant sequence. Thus the mutant protein does not contain any of the four conserved domains that play a major role in the function of the protein. Analysis of the 2000 nucleotides up-stream the start codon of ORF4 revealed the presence of three Ethylene Responsive Elements (ERE; AWTTCAAA) at positions −667, −700 and −1955 relative to the start codon, one bZIP protein binding motif (TGACG) at −1632 and one G-box (CACGTG) at −1329. The presence of ERE motifs, bZIP protein binding motifs and G-box in the promoter sequence of ORF4 suggests that the transcription of ORF4 is stimulated by ethylene and jasmonate.

### 2.6 Expression of ORF4 in Anther, Fruit and Other Tissues

We tested the presence of ORF4 transcript in several tissues including abscission zones of leaf and flowers, mature fruits and anthers at anthesis. No ORF4 transcript was detected in the abscission zones (FIG. 7). The presence of ORF4 transcript was confirmed in anthers as well as in the fruit. In order to study the evolution of the transcription level of ORF4 over different stages of anther and fruit development, we performed a quantitative expression analysis of ORF4 at four developmental stages of anthers: flower bud; pre-anthesis; anthesis; post-anthesis, and eight developmental stages of the fruit, from five dap (days after pollination) to 57 dap (mature fruit); 47 dap corresponded to breaker stage (data not shown). In anthers, the transcription level of ORF4 was tested on Moneymaker and ps-2ABL, and in the fruits only on Moneymaker. No ORF4 transcript was detected in the fruit before 37 dap. From 37 dap ORF4 transcript was detected and increased significantly over time to reach a maximum at mature stage (57dap). In anthers of Moneymaker, ORF4 transcripts were detected already at flower bud stage. At pre-anthesis the level of ORF4 transcript was similar to flower bud. ORF4 transcript accumulation increased at anthesis and reached a maximum at post-anthesis. In anthers of ps-2ABL, ORF4 transcript was also detected, except at flower bud stage. However this transcript level in ps-2ABL anthers was significantly lower than in Moneymaker anthers at anthesis and post-anthesis.

### 3. Discussion

The ps-2 Advanced Breeding Line produces anthers that do not undergo dehiscence. In this study we showed that anther development/dehiscence of ps-2ABL is blocked in the ultimate phase. The stomium does not degenerate and the endothecium wall does not thicken. In absence of these two physiological changes, the anther remains closed and the epidermal cells lack the rigidity that could eventually break the stomium and liberate the pollen.

This phenotypic mutation is recessive and under the control of one single locus. In a previous study we fine mapped the ps-2 gene on the short arm of Chromosome 4 (Gorguet et al, 2006). Here we report the isolation and functional characterization of the ps-2 gene. We found that the ps-2 phenotype is the result of a single nucleotide mutation in a polygalacturonase gene unknown to date, composed of nine exons. This single nucleotide mutation is located on the last nucleotide of the 3′ end of the fifth exon, affecting the intron splicing recognition site, which is changed from CAG/GTATCG to CAC/GTATCG (exon 3′/intron 5′). Though the Cytosine base is met in 11% of the intron splice sites at this specific position in plants, the combination “CAC” at the exon 3′end has never been detected in any splice site in plants (Brown 1986). The 5^{th} exon is spliced out together with the two flanking introns. Analysis of Arabidopsis mutants with mutations around splice sites has revealed several examples of exon skipping in plant splicing (reviewed by Brown and Simpson, 1998). Most of these mutations are located in the intron part of the recognition splice sites. To our knowledge, in plants, the only mutant showing exon skipping due to a mutation in the exon part of a recognition splice site, to date, was the spy-1 mutant in Arabidopsis (Jacobsen et al, 1996) in which the CAG/GTTTGA (exon 3′/intron 5′) recognition splice site at the end of the eight exon was mutated into CAA/GTTTGA. The exon skipping observed in the mutated allele of ORF4 induces a frame-shift in the rest of the sequence, which has for consequence to create an early stop codon 14 aa further. The complete mutant protein is therefore 154 aa long in comparison to 392 aa long for the wild type, and do not contain any of the four domains characteristic of PGs.

### 3.1 ps-2 is a PG of Clade B

The isolated gene responsible for the ps-2 phenotype is a PG unknown to date. We propose the acronym DPG, standing for Dehiscence PolyGalacturonase, or in the case of the tomato gene TDPG, standing for Tomato Dehiscence PolyGalacturonase. Phylogenetic analysis of TDPG revealed a close similarity with PG of clade B as defined by Hadfield and Bennett (1998). Clade B is here composed of fruit PGs, among them the Tomato Fruit PG, as well as silique or pod dehiscence PGs. Other tomato PGs cluster in different clades. In accordance to the assertion that the divergence of PG gene families occurred prior to the separation of the angiosperm species (Hadfield and Bennett, 1998), TDPG is here more closely related to genes of the same clade, from other species, than to tomato PGs from other clades. Most of the other tomatoes PGs are related to abscission (TAPG). In our study, expression of TDPG was not detected in flower and leaf abscission zones. However, in addition to anther tissues, we detected mRNA transcript of TDPG in fruits.

### 3.2 TDPG Transcript Accumulation Increases Along with the Development of the Anthers

We measured the relative level of TDPG transcript at different stages in anthers. TDPG transcript is already detected in anthers of Moneymaker at flower bud stage. The transcript level increases over stages to reach a maximum at post-anthesis, when anthers are dehisced. This increase of TDPG transcript accumulation is parallel to the septum and stomium degeneration as well as the thickening of the endothecial cell wall observed in the anthers. TDPG transcript level in ps-2ABL anthers was detected from pre-anthesis on, but the transcript level remained very low in comparison to Moneymaker, at anthesis and post-anthesis. Very likely the mutant mRNA is recognized as non-sense and degraded by Nonsense-mediated mRNA decay (NMD). NMD functions as a quality control mechanism to eliminate abnormal transcripts (Lejeune and Maquat 2005).

### 3.2 TDPG is Likely Under the Control of Ethylene and Jasmonate

The presence of ethylene and jasmonate responsive elements in the promoter region of TDPG suggests that the transcription of DPG genes could be induced by both hormones. Ethylene has already been involved in the timing of anther dehiscence in tobacco (Rieu et al, 2003). More recently, in petunia, it has been shown that ethylene regulates the synchronization of anther dehiscence with flower opening (Wang and Kumar, 2006). In addition, many studies already identified jasmonate as a key compound in the process and timing of anther dehiscence. Several mutants in JA biosynthetic enzymes have been identified for the study of this phenomenon. Scott et al (2004) have suggested that ethylene and JA may act redundantly in the control of anther dehiscence, which would explain why Arabidopsis mutants such as dde-1, which cannot synthesize JA within the stamens, or the ethylene insensitive Tetr mutant, eventually undergo anther dehiscence.

In the present study we showed that anthers of ps-2ABL remain indehiscent, in contrast to delayed dehiscence in the other mutants, which strengthen the hypothesis that DPG acts down-stream of Ethylene and JA in the control of anther dehiscence and that DPG is the main actor of this process.

### 3.3 TDPG May Also Play a Role in Tomato Fruit Ripening:

TFPG, the only tomato fruit polygalacturonase identified to date, has been characterized as one of the main actors in the process of fruit softening. Anti-sense repression of TFPG has lead to the production of tomato fruit with longer shelf life but the fruits did undergo ripening indicating that other actors also play a relevant role in the process of fruit softening (Smith et al, 1988). DPGs may well be one of these actors by contributing to the fruit cell wall degradation. TDPG transcript was detected in the late stages of fruit development in Moneymaker (FIG. 8). Maximum transcripts were found at mature stage (57dap). Similarly TFPG has been detected only at ripening stages, starting at mature green or at breaker stage (Thompson et al, 1999; Eriksson et al, 2004). The level of TFPG was also found to increase over time from breaker stage to mature fruit, in cv. AC and Liberto (Thompson et al, 1999).

In tomato, although the regulation of PG mRNA accumulation by ethylene remained for a long time ambiguous, it has been demonstrated that TFPG accumulation was ethylene dependent in the process of fruit ripening (Sitrit and Bennett, 1998). In accordance ERE motifs have been found in the promoter of TFPG (Montgomery et al, 1993). Ethylene is presented as the major plant hormone in the control of fruit ripening (reviewed by Giovannoni, 2004). Expression of anti-sense RNA to the rate limiting enzyme in the biosynthetic pathway of ethylene inhibits fruit ripening in tomato and as consequence down regulates the production of TFPG (Oeller et al, 1991; Sitrit and Bennett, 1998).

Earlier in this study we suggested that DPG was under the control of ethylene due to the presence of ERE motifs in the promoter sequence. Repression of ethylene in the tomato fruit is likely to inhibit both TFPG and TDPG and therefore prevents completely the process of fruit ripening. A simplified model for the hormonal control of DPG in anther dehiscence and fruit maturation is presented in FIG. 8.

It is not clear whether the role of DPG in fruit maturation and shelf life is of similar importance than TFPG. The comparison between ps-2ABL after manual opening of the anthers and Moneymaker or any other normal tomato line is unreliable due to the difference of genetic background. Knock out of DPG by anti-sense RNA or RNAi in a normal tomato line could answer to that question. Time between hand pollination to mature fruit stage and fruit shelf life could be measured and compared to the untransformed control in order to evaluate the effect of DPG in the fruit. Knock out of homologs of TDPG in other plants species is also of valuable interest to verify whether the control of anther dehiscence it its ultimate phase is conserved among species.

TABLE 3
Alignment of the Wild Type sequence (WT) and the Mutant sequence

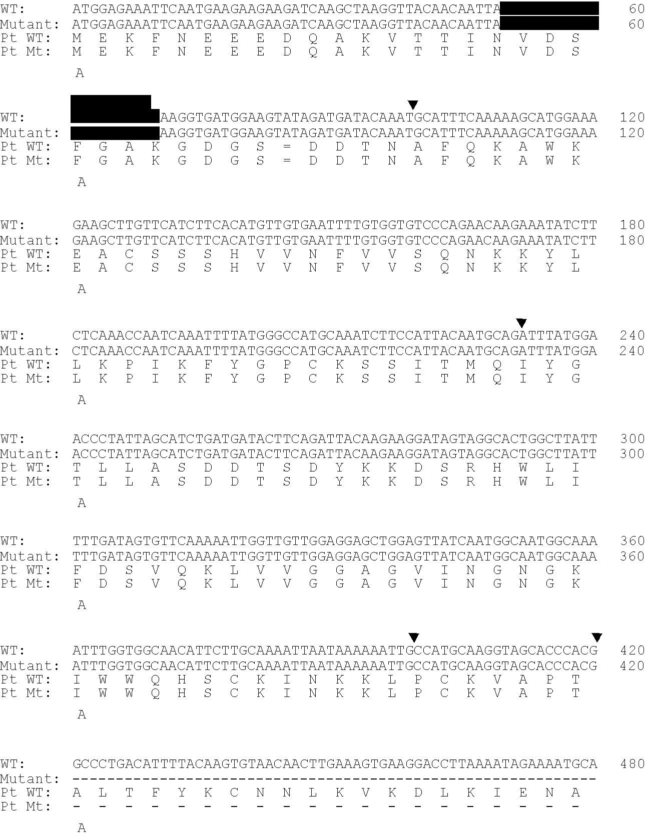

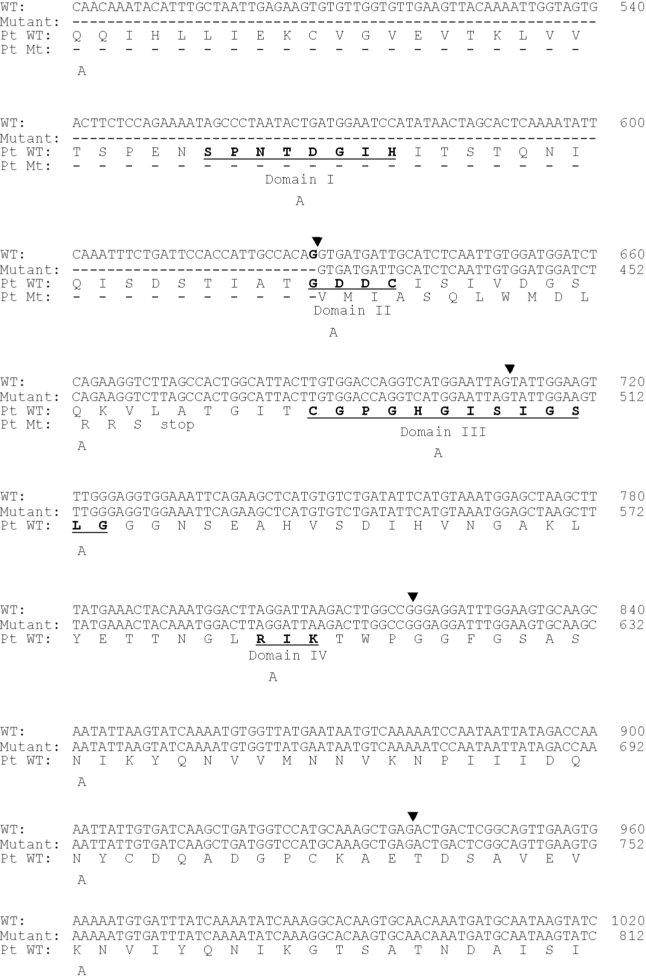

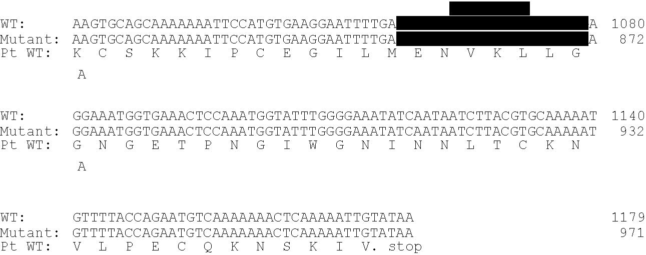

Pt WT: Protein sequence of the Wild Type (Moneymaker)
Pt Mt: Protein sequence of the Mutant (ps-2ABL)
The nucleotide “G” in bold indicates the location of the mutation
Conserved protein domains are indicated in bold underlined
Positions of the introns are indicated with a black arrow above the sequence ▾
Positions of the primers sequences used to amplify the quasi-complete coding sequence are indicated in gray

### REFERENCES

Atkinson R G and Gardner R C (1993) A polygalacturonase gene from kiwifruit (Actinidia deliciosa) Plant Physiol. 103:669-670
Atkinson R G (1994) A cDNA clone for endopolygalacturonase from apple. Plant Physiol. 105: 1437-1438
Bergey D R, Orozco-Cardenas M, de Moura D S, Ryan C A (1999) A wound- and systemin-inducible polygalacturonase in tomato leaves Proc. Natl. Acad. Sci. U.S.A. 96 (4), 1756-1760 (1999)
Bird C R, Smith C J, Ray J A, Moureau P, Bevan M W, Bird A S, Hughes S, Morris P C, Grierson D, Schuch W (1988) The tomato polygalacturonase gene and ripening-specific expression in transgenic plants Plant Mol. Biol. 11: 651-662
Brown J W S (1986) A catalogue of splice junction and putative branch point sequences from plant introns. Nucleic acids research 14: 9549-9559
Brown J W S and Simpson C G (1998) Splice site selection in plant pre-mRNA splicing Annual review of plant physiology and plant molecular biology 49:77-95
Brugmans B, Hutten R G B, Rookmaker A N O, Visser R G F, Van Eck H J (2006) Exploitation of a marker dense linkage map of potato for positional cloning of a wart disease resistance gene. Theoretical and Applied Genetics 112:269-277
Budiman M A, Mao L, Wood T C, Wing R A (2000) A deep-coverage tomato BAC library and prospects toward development of an STC framework for genome sequencing. Genome Res 10:129-136
Christiansen L C, Dal Degan F, Ulvskov P, Borkhardt B (2002) Examination of the dehiscence zone in soybean pods and isolation of a dehiscence-related endopolygalacturonase gene Plant Cell Environ. 25:479-490
Colbert T, Till B J, Tompa R, Reynolds S, Steine M N, Yeung A T, McCallum C M, Comai L, Henikoff S. High-throughput screening for induced point mutations. Plant Physiol. 2001 June; 126(2):480-4
Dornelas M, van Lammeren A, and Kreis M (2000). Arabidopsis thaliana SHAGGY-related protein kinases (AtSK11 and 12) function in perianth and gynoecium development. Plant Journal 21: 419-429
Dubald M, Barakate A, Mandaron P, Mache R (1993) The ubiquitous presence of exopolygalacturonase in maize suggests a fundamental cellular function for this enzyme. The plant journal 4: 781-791
Gorguet B, Schipper D, van Heusden A W, Lindhout P (2006) High resolution fine mapping of ps-2, a mutated gene conferring functional male sterility in tomato due to non-dehiscent anthers. Theoretical and Applied Genetics. 113:1437-1448
Hadfield K A, Bennett A B (1998) Polygalacturonases: Many genes in search of a function. Plant physiology 117: 337-343
Higgins D, Thompson J, Gibson T, Thompson J D, Higgins D G, Gibson T J (1994) CLUSTAL W: improving the sensitivity of progressive multiple sequence alignment through sequence weighting, position-specific gap penalties and weight matrix choice. Nucleic Acids Res. 22:4673-4680
Hong S B and Tucker M L (1998) Genomic organization of six tomato polygalacturonases and 5′ upstream sequence identity with tap1 and win2 genes Mol. Gen. Genet. 258: 479-487
Hong S-B and Tucker M L, (2000) Molecular characterization of a tomato polygalacturonase gene abundantly expressed in the upper third of pistils from opened and unopened flowers. Plant Cell Reports 19: 680-683
Huala E, Dickerman A W, Garcia-Hernandez M, Weems D, Reiser L, LaFond F, Hanley D, Kiphart D, Zhuang M, Huang W, Mueller L A, Bhattacharyya D, Bhaya D, Sobral B W, Beavis W, Meinke D W, Town C D, Somerville C, Rhee S Y (2001) The Arabidopsis Information Resource (TAIR): a comprehensive database and web-based information retrieval, analysis, and visualization system for a model plant. Nucleic Acids Res. 29:102-105
Jacobsen S E, Binkowski K A, Olszewski N E (1996) SPINDLY, a tetratricopeptide repeat protein involved in gibberellin signal transduction in Arabidopsis. Proceedings of the National Academy of Sciences of the United States of America 93: 9292-9296
Jenkins E S, Paul W, Coupe S A, Bell S, Davies L, Roberts J A. Characterization and expression of an mRNA encoding polygalacturonase during pod development in oilseed rape (Brassica napus L.) Unpublished
Keijzer C J (1987) The processes of anther dehiscence and pollen dispersal. I. The opening mechanism of longitudinally dehiscing anthers. New Phytologist 105: 487-498
Lejeune F, Maquat L E (2005) Mechanistic links between nonsense-mediated mRNA decay and pre-mRNA splicing in mammalian cells. Current Opinion in Cell Biology 17: 309-315
Lester D R, Speirs J, Orr G, Brady C J (1994) Peach (Prunus persica) endopolygalacturonase cDNA isolation and mRNA analysis in melting and nonmelting peach cultivars Plant Physiol. 105: 225-231
Markovic O, Janecek S (2001) Pectin degrading glycoside hydrolases of family 28: sequence-structural features, specificities and evolution. Protein engineering 14: 615-631
McCallum C M, Comai L, Greene E A, Henikoff S. Targeted screening for induced mutations. Nat Biotechnol. 2000 April; 18(4):455-7
McCallum C M, Comai L, Greene E A, Henikoff S. Targeting induced local lesions IN genomes (TILLING) for plant functional genomics. Plant Physiol. 2000a June; 123(2):439-42
Montgomery J, Pollard V, Deikman J, Fischer R L (1993) Positive and negative regulatory regions control the spatial-distribution of polygalacturonase transcription in tomato fruit pericarp. The plant cell 5: 1049-1062
Mueller L A, Solow T H, Taylor N, Skwarecki B, Buels R, Binns J, Lin C, Wright M H, Ahrens R, Wang, Y, Herbst E V, Keyder E R, Menda N, Zamir D, Tanksley S D (2005) The SOL Genomics Network. A comparative resource for Solanaceae biology and beyond. Plant Physiol 138:1310-1317
Nunan K J, Davies C, Robinson S P and Fincher G B (2001) Expression patterns of cell wall-modifying enzymes during grape berry development. Planta 214:257-264
Oeller P W, Min-Wong L, Taylor L P, Pike D A, Theologis A (1991) Reversible inhibition of tomato fruit senescence by antisense RNA. Science 254: 437-439
Ogasawara S and Nakajima T. A gene coding for bell pepper endo-polygalacturonase is expressed during fruit ripening. Unpublished
Petersen M, Sander L, Child R, van Onckelen H, Ulvskov P, Borkhardt B (1996) Isolation and characterisation of a pod dehiscence zone-specific polygalacturonase from Brassica napus. Plant Mol. Biol. 31: 517-527
Rao M N, Kembhavi A A, Pant A (1996) Implication of tryptophan and histidine in the active site of endo-polygalacturonase from Aspergillus ustus: elucidation of the reaction mechanism. Biochim. Biophys. Acta 1296: 167-173
Rieu I, Wolters-Arts M, Derksen J, Mariani C, Weterings K (2003) Ethylene regulates the timing of anther dehiscence in tobacco. Planta 217: 131-137
Rodriguez-Gacio M, Nicolas C, Matilla A (2004) Cloning and analysis of a cDNA encoding an endo-polygalacturonase expressed during the desiccation period of the silique-valves of turnip-tops (Brassica rapa L. cv. Rapa) Plant Physiol. 161: 229-236
Rouster J, Leah R, Mundy J, Cameron-Mills V (1997) Identification of a methyl jasmonate-responsive region in the promoter of a lipoxygenase 1 gene expressed in barley grain. The plant journal 11: 513-523
Sander L, Child R, Ulvskov P, Albrechtsen M, Borkhardt B (2001) Analysis of a dehiscence zone endo-polygalacturonase in oilseed rape (Brassica napus) and Arabidopsis thaliana: evidence for roles in cell separation in dehiscence and abscission zones, and in stylar tissues during pollen tube growth. Plant Mol. Biol. 46: 469-479
Scott R J, Spielman M, Dickinson H G (2004) Stamen structure and function. The plant cell 16: S40-S60
Sitrit Y, Hadfield K A, Bennett A B, Bradford K J, Downie A B (1999) Expression of a polygalacturonase associated with tomato seed germination Plant Physiol. 121: 419-428
Smith C J S, Watson C F, Ray J, Bird C R, Morris P C, Schuch W, Grierson D (1988) Anti-sense RNA inhibition of polygalacturonase gene expression in transgenic tomatoes. Nature 334: 724-726
Swofford D L (2002) PAUP*. Phylogenetic Analysis Using Parsimony (*and Other Methods). Version 4. Sinauer Associates, Sunderland, Mass.
Torki M, Mandaron P, Mache R, Falconet D (2000) Characterization of a ubiquitous expressed gene family encoding polygalacturonase in Arabidopsis thaliana. Gene 242:427-436
Wang Y, Kumar P P (2006) Characterization of two ethylene receptors PhERS1 and PhETR2 from petunia: PhETR2 regulates timing of anther dehiscence. Journal of Experimental Botany doi:10.1093/jxb/er1229

1. A nucleic acid molecule comprising a nucleotide sequence encoding a DPG polypeptide with polygalacturonase activity, wherein the nucleotide sequence is selected from the group consisting of:
(a) a nucleotide sequence encoding an amino acid sequence that has at least 60% sequence identity with the amino acid sequence of SEQ ID NO. 2;
(b) a nucleotide sequence that has at least 55% sequence identity with the nucleotide sequence of SEQ ID NO. 1;
(c) a nucleotide sequence the complementary strand of which hybridises to a nucleotide sequence of (a) or (b); and,
(d) a nucleotide sequence the sequence of which differs from the sequence of a nucleotide sequence of (c) due to the degeneracy of the genetic code.

2. A nucleic acid molecule comprising a nucleotide sequence of at least 26 contiguous nucleotides from SEQ ID NO: 1.

3. A method for detecting, isolating, amplifying and/or analysing a DPG allele in a plant, the method comprising the step of providing a sample comprising nucleic acids of the plant and hybridising the nucleic acids of the plant with a nucleic acid molecule comprising a nucleotide sequence of at least 10 contiguous nucleotides from a nucleotide sequence as defined in claim 1.

4. A method according to claim 3, wherein the DPG allele is a ps-2-allele.

5. A method according to claim 4, wherein the ps-2-allele is an allele that has a C, A or T as last nucleotide of the 3′ end of the fifth exon of the DPG gene, preferably the allele has a C as last nucleotide of the 3′ end of the fifth exon of the DPG gene.

6. A method of marker-assisted breeding comprising contacting a plant cell with a nucleotide sequence of at least 10 contiguous nucleotides from a nucleotide sequence as defined in claim 1.

7. A method according to claim 6, wherein the marker-assisted breeding comprises the detection of a ps-2-allele.

8. A method according to claim 7, wherein the ps-2-allele is an allele that has a C, A or T as last nucleotide of the 3′ end of the fifth exon of the DPG gene, preferably the allele has a C as last nucleotide of the 3′ end of the fifth exon of the DPG gene.

9. A method for producing a plant with non-dehiscent anthers, wherein the method comprises the steps of:
a) crossing a first plant with a second plant that is homozygous for a ps-2-allele;
b) backcrossing the F1 generation and further generations for at least two generation with the first plant as recurrent parent; and,
c) selfing the furthest backcrossed generation obtained in b) for at least one-generations;
wherein a molecular marker is used in at least one of steps b) and c) to select for a plant that is homozygous for the ps-2-allele.

10. A method according to claim 9, wherein the molecular marker is a marker specific for a DPG allele, which marker is present within the genome of the plant no more than 100 kb from a nucleotide sequence encoding the polypeptide with polygalacturonase activity as defined in claim 1 or a part thereof.

11. A method according to claim 10, wherein the molecular marker the molecular marker is or detects a C, A or T as last nucleotide of the 3′ end of the fifth exon of the DPG gene, of which a C as last nucleotide of the 3′ end of the fifth exon of the DPG gene is most preferred.

12. A method for producing a plant with a mutation in a DPG-allele, wherein the method comprises the steps of:
a) mutagenising seeds of a plant complex;
b) growing plants of the mutagenised seeds obtained in a);
c) optionally, backcrossing the plants obtained in b) for at least one generation; and,
d) screening plants obtained in b) or c) for the presence of a mutation in a DPG-allele.

13. A method according to claim 12, wherein the mutation in the DPG-allele cause the allele to be a ps-2-allele.

14. A method according to claim 13, wherein the ps-2-allele is an allele that has a C, A or T as last nucleotide of the 3′ end of the fifth exon of the DPG gene, preferably the allele has a C as last nucleotide of the 3′ end of the fifth exon of the DPG gene.

15. A method for producing a transgenic plant with non-dehiscent anthers, wherein the method comprises the step of transforming a plant cell with a nucleic acid construct comprising at least a fragment of a nucleotide sequence encoding a DPG as defined in claim 1, or a complement thereof, wherein presence of the nucleic acid construct in a cell of the plant reduces expression of DPG activity to a level that effects positional sterility and non-dehiscent anthers.

16. A method according to claim 15, wherein the nucleotide sequence is operably linked to a promoter for expression in a plant cell and wherein the expression of the nucleotide sequence reduces expression of DPG activity by RNA interference.

17. A method according to claim 15, wherein nucleic acid construct is a construct for homologous recombination and wherein the nucleotide sequence comprises a mutation that reduces expression of DPG activity to a level that effects positional sterility and non-dehiscent anthers.

18. A nucleic acid construct comprising at least a fragment of a nucleotide sequence encoding a DPG as defined in claim 1, or a complement thereof, operably linked to a promoter for expression in a plant cell.

19. A nucleic acid construct comprising at least a fragment of a nucleotide sequence encoding a DPG as defined in claim 1, or a complement thereof, operably linked to a promoter for expression in a plant cell, wherein the fragment comprises a sequence of 30 contiguous nucleotides from a nucleotide sequence having at least 60% sequence identity to a nucleotide sequence encoding a DPG as defined in claim 1, or a complement thereof.
